(19) United States
(12) Patent Application Publication
(10) Pub. No.: US 2013/0053592 A1
(43) Pub. Date: Feb. 28, 2013

(54) FATTY ACIDS AS CO-CATALYSTS IN PREPARATION OF GLYCERIDES FROM ETHYL ESTERS

(71) Applicant: Asgeir Saebo, Eidsnes (NO)

(72) Inventor: Asgeir Saebo, Eidsnes (NO)

(73) Assignee: PHARMA MARINE AS, SOEVIK (NO)

(21) Appl. No.: 13/597,756

(22) Filed: Aug. 29, 2012

Related U.S. Application Data
(60) Provisional application No. 61/529,579, filed on Aug. 31, 2011.

(51) Int. Cl. C12P 7/64; C09K 3/00; C07C 69/587

(52) U.S. Cl. 554/224; 435/134; 252/183.11

(57) ABSTRACT

Methods of producing concentrates of omega-3 fatty acid ethyl esters, from an ethyl ester composition comprising use of free fatty acids as co-catalysts for enzymatic synthesis of partial glycerides are provided. In some embodiments, methods of producing concentrates of ethyl-eicosapentanoate and/or ethyl-docosahexanoate from an ethyl ester composition containing the esters comprise a) adding glycerol, an immobilized enzyme catalyst, and a free fatty acid co-catalyst b) allow glyceryl ester synthesis to proceed by adjustment of temperatures, time and vacuum such that a substantial amount of partial glycerides is formed and c) separating an ethyl ester fraction enriched in ethyl eicosanoate and ethyl docosahexanoate from the glyceride mixture.

### CROSS-REFERENCE TO RELATED APPLICATIONS

This application claims priority to pending U.S. Provisional Patent Application No. 61/529,579, filed Aug. 31, 2011, the contents of which are incorporated by reference in their entireties.

### FIELD OF THE INVENTION

The present invention relates to methods for producing products containing fatty acid moieties, and in particular to products containing omega-3 fatty acid moieties.

### BACKGROUND OF THE INVENTION

Fatty acid ethyl ester is the preferred form in which omega-3 and other highly unsaturated and fatty acids are purified and concentrated. Alternatively free fatty acids can be distilled, but stability and distillation performance is far better for ethyl esters. Various enzymatically catalyzed reactions have been proposed for the purification of omega-3 and other fatty acids. Enzymes that more easily catalyze reactions with saturated and oligo-unsaturated fatty acids than highly unsaturated ones are particularly of interest.

Unfortunately, such enzymes working perfectly with free fatty acids, do often not function with ethyl esters. This has been demonstrated in U.S. Pat. No. 6,518,049. This patent teaches the use of immobilized enzymes for selectively partial esterification of free fatty acids to glycerol. Saturated and oligo-unsaturated fatty acids were much more readily esterified than EPA and particularly DHA and thus a mixture of EPA and DHA in free fatty acid form together with partial glycerides of saturated and oligo-unsaturated fatty acids could be obtained. By subsequent distillation a free fatty acid mixture with enhanced content of EPA and DHA could be obtained. Ethyl esters were tested with very poor yield, and the inventors concluded: “This experiment shows that ethyl esters, although more advantageous and more readily available as raw materials, do not represent a preferred starting material in this invention.”

To overcome the problem of slow reaction with ethyl esters, another group investigated the careful addition of water vapor during esterification. In their pending application U.S. 2006/0286266 it is shown that a concentrate of DHA in ethyl ester form was easily made.

Based on the facts above it was anticipated that the enzymes used would work well as a tool to remove free fatty acids from a blend of free fatty acids and ethyl esters. It was assumed that since only free fatty acids were shown to easily esterify to glycerol, the free fatty acids could be separated from the ethyl ester fraction by rapid esterification to glycerol. This did to some extent occur, but to our great surprise, fatty acid ethyl esters in saturated and mono-unsaturated form also were very readily esterfied to glycerol. Apparently, the water liberated from the esterification of free fatty acids somehow are retained by the enzyme at the site of reaction and readily allow esterification also of ethyl esters.

This technology opens the avenue for use of free fatty acids as co-catalysts for reacting ethyl esters with glycerol and opens up a new avenue for interesting product blends. The fatty acid acting as a co-catalyst should preferably be removed from the finished product, but if a bioactive fatty acid of interest to blend into a concentrate of EPA/DHA is used, the free fatty acid used as a catalyst may preferably be left in the product.

### SUMMARY OF THE INVENTION

The present invention relates to methods for producing products containing fatty acid moieties, and in particular to products containing omega-3 fatty acid moieties.

In some embodiments, the present invention provides methods of producing concentrates of omega-3 fatty acid ethyl esters, from an ethyl ester composition comprising use of free fatty acids as co-catalysts for enzymatic synthesis of partial glycerides. In some embodiments, the methods further comprise use of fatty acids with carbon chain length of C 6 to C 30. In some embodiments, the methods further comprise use of free fatty acids obtained from the ethyl ester itself by partial hydrolysis of the ethyl ester mixture. In some embodiments, the methods further comprise use of a bioactive fatty acid. In some embodiments, the bioactive fatty acid is selected from the group consisting of alpha linolenic acid, gamma linolenic acid, conjugated fatty acids, 3-thia fatty acids, short and medium chain fatty acids, palmitoleic acid, and pinolenic acid. In some embodiments, the present invention provides a triglyceride product containing less than 50% diglycerides made using the foregoing methods.

In some embodiments, the present invention provides a lipid composition comprising an ethyl ester fraction A and a free fatty acid fraction B, wherein fraction A makes up 70-99% of the composition and fraction B makes up 1-30% of said composition, wherein fraction A comprises greater than 50% eicosapentaenoic and docosahexaenoic acid moieties and fraction B comprises less than 30% eicosapentaenoic and docosahexaenoic acid moieties. In some embodiments, the present invention provides a lipid composition containing greater than 30% eicosapentaenoic and docosahexaenoic acid moieties, the composition characterized in comprising a mixture of partial glycerides, ethyl esters and free fatty acids.

In some embodiments, the present invention provides methods of producing concentrates of ethyl-eicosapentanoate and/or ethyl-docosahexanoate from an ethyl ester composition containing said esters comprising a) adding glycerol, an immobilized enzyme catalyst, and a free fatty acid co-catalyst b) allow glyceryl ester synthesis to proceed by adjustment of temperatures, time and vacuum such that a substantial amount of partial glycerides is formed and c) separate ethyl ester fraction enriched in ethyl eicosanoate and ethyl docosahexanoate from the glyceride mixture.

### DETAILED DESCRIPTION OF THE INVENTION

To an ethyl ester fraction, preferably partly purified by molecular distillation and being the subject of further concentration of EPA and DHA, a free fatty acid is added in the amount of about 20-50% in the presence of a lipase. The lipase may position specific or non-specific. This acid can for instance be oleic acid or another fatty acid. If oleic acid or a blend of vegetable fatty acids are used, those acids will partly remain in the finished product unless additional techniques are used for removal. Fatty acids of short to medium chain can easily be removed in the further purification step anyhow required. However, also, short and medium chain fatty acids are of interest to supplement market and therefore a product can be made where said fatty acids are retained in the product. By adjusting ratio of glycerol to ethyl esters/free fatty acids and adjusting the vacuum and reaction time, a mixture is made where diacyl and triacyl glycerols preferably reaches a level of about 50-60%. Then, by molecular distillation, non-esterified ethyl esters and minor amounts (about 5-20%) of free fatty acids are collected. The free fatty acids can be removed by appropriate technology or optionally, the acids can be a part of the product if desired, particularly if the free fatty acid co-catalyst is a desirable functional fatty acid. The product may be further refined to obtain a product in triglyceride form.

### EXPERIMENTAL

### Example 1

174 gram of an ethyl ester composition containing about 50 mg EPA and about 500 mg DHA/gram was mixed with 174 gram of a conjugated linoleic acid product. Then 26 grams of glycerol and 2 grams of immobilized enzyme RM-IM was added to the mixture. The mixture was stirred under vacuum at 60 C for 24 hours. The free fatty acid content was reduced to 6.3% by the end of the reaction. The ethyl ester fraction comprised 33.6%, monoglycerides 2.5%, diglycerides 40.0%, and triglycerides 17.6%. The ethyl ester fraction contained 64% DHA, 8% EPA and 17% CLA.

1. A method of producing concentrates of omega-3 fatty acid ethyl esters from an ethyl ester composition comprising:
mixing an ethyl ester fraction with free fatty acids in the presence of a lipase and glycerol under conditions such that partial glycerides are formed; and
separating ethyl esters from said mixture.

2. The method of claim 1, further comprising use of fatty acids with carbon chain length of C 6 to C 30.

3. The method of claim 1, further comprising use of free fatty acids obtained from the ethyl ester itself by partial hydrolysis of the ethyl ester mixture.

4. The method claims 1, further comprising use of a bioactive fatty acid.

5. The method of claim 4, wherein aid bioactive fatty acid is selected from the group consisting of alpha linolenic acid, gamma linolenic acid, conjugated fatty acids, 3-thia fatty acids, short and medium chain fatty acids, palmitoleic acid, and pinolenic acid.

6. A product made according to claim 1, further processed to yield a triglyceride product containing less than 50% diglycerides.

7. A lipid composition comprising an ethyl ester fraction A and a free fatty acid fraction B, wherein fraction A makes up 70-99% of said composition and fraction B makes up 1-30% of said composition, wherein fraction A comprises greater than 50% eicosapentaenoic and docosahexaenoic acid moieties and fraction B comprises less than 30% eicosapentaenoic and docosahexaenoic acid moieties.

8. A lipid composition containing greater than 30% eicosapentaenoic and docosahexaenoic acid moieties, said composition characterized in comprising a mixture of partial glycerides, ethyl esters and free fatty acids.

9. A method of producing concentrates of ethyl-eicosapentanoate and/or ethyl-docosahexanoate from an ethyl ester composition containing said esters comprising:
mixing glycerol, an immobilized enzyme catalyst, and a free fatty acid co-catalyst;
allowing glyceryl ester synthesis to proceed by adjustment of temperature, time and vacuum such that a substantial amount of partial glycerides is formed; and
separating an ethyl ester fraction enriched in ethyl eicosanoate and ethyl docosahexanoate from the glyceride mixture.
